(19) United States
(12) Patent Application Publication
(10) Pub. No.: US 2024/0404088 A1
(43) Pub. Date: Dec. 5, 2024

(54) PROCESS AND DEVICE FOR ANALYZING A TEXTURE OF A TISSUE

(71) Applicant: MEDIMAPS GROUP SA, Plan-les-Ouates (CH)

(72) Inventors: Didier HANS, Genthod (CH); Franck MICHELET, PESSAC (FR); Lionel BEAUGÉ, Saint Julien en Genevois (FR); Guillaume GATINEAU, Saint Julien en Genevois (FR)

(73) Assignee: MEDIMAPS GROUP SA, Plan-les-Ouates (CH)

(21) Appl. No.: 18/694,770

(22) PCT Filed: Sep. 29, 2022
(86) PCT No.: PCT/EP2022/077236

§ 371 Date: Mar. 22, 2024

(30) Foreign Application Priority Data
Sep. 29, 2021 (WO) PCT/EP2021/076863

(51) Int. Cl. G06T 7/40

(52) U.S. Cl. CPC G06T 7/40; G06T 2207/10116; G06T 2207/30004

(57) ABSTRACT

A process for analyzing a texture of a human or animal tissue from a digitized image, obtained by X-Ray based imaging system. The process includes a step of calculating at least one texture score B for the image by applying an experimental variogram to the tissue texture. Also, a device for analyzing the texture of a human or animal tissue from the digitized image.

### FIELD

The present invention relates to a process and a device for analyzing a texture of a human or animal tissue from a digitized image.

The invention also relates to, but is not limited to, medical in relation to bone health, medical x-ray-based imaging, primary and secondary osteoporosis, and bone fragility fracture.

### BACKGROUND

In early nineties, the World Health Organization (WHO) defines osteoporosis conceptually as a systemic skeletal disease characterized by low bone mass (decreased quantity) and microarchitectural deterioration of bone tissue (decreased quality) with a consequent increase in bone fragility and susceptibility to fracture (Consensus development conference: diagnosis, prophylaxis, and treatment of osteoporosis. Am J Med 94, 646-650 (1993)). It has been further defined early 2000 by the NIH-National Institutes of Health-(Osteoporosis prevention, diagnosis, and therapy. Jama. 2001; 285(6): 785-95.) as a skeletal disorder characterized by compromised bone strength predisposing to an increased risk of fracture. In essence, in osteoporosis, the deteriorated bone strength leads to the traumatic outcome of fragility fracture.

Bone strength reflects the integration of two main features: bone quantity (i.e. bone density) and bone quality. Bone density is expressed as grams of mineral per area or volume and in any given individual is determined by peak bone mass and amount of bone loss. Bone quality refers to bone architecture, turnover, damage accumulation (e.g., microfractures) and mineralization. Bone architecture is a generic term used for many different entities and can be further refined. At the macroscopic level, bone architecture, known as bone macro-structure (also referred as bone macro-architecture) describes the overall shape and geometry of bone as well as the differentiation into cancellous (also referred to as trabecular) and cortical bone. Most typical parameters describing macro-architecture are cortical thickness, moment of inertia and other geometrical measures. At the microscopic level, i.e., usually if the spatial resolution of the acquired images is better than 100 μm, bone architecture, known as bone micro-structure (also referred as bone microarchitecture) can be described by a combination of parameters such as trabecular thickness and separation of trabeculae, trabecular number, trabecular connectivity and parameters comprehensively describing the network architecture, such as the structure model index (SMI) (Macro- and Microimaging of Bone Architecture, Klaus Engelke, Sven Prevrhal, Harry K. Genant, in Principles of Bone Biology (Third Edition) Volume II, 2008, Pages 1905-1942. Editors: John Bilezikian Lawrence Raisz T. John Martin—Publisher: Academic Press. Published Date: 29th September 2008).

Based on its etiology, osteoporosis can be primary or secondary. Primary or age-related osteoporosis is the most common type (Mirza F, Canalis E. Management of endocrine disease: Secondary osteoporosis: pathophysiology and management. European journal of endocrinology. 2015; 173(3): R131-51). Physiologically, the peak bone mass is typically reached by the age 30 in both women and men; afterwards, women lose bone more rapidly than men and this bone loss is more obvious post-menopause due to the estrogen deficiency. That is why this type of osteoporosis is more present in women and is also known as postmenopausal osteoporosis. Its main cause is sex hormones deficiency due to the advancing age.

Secondary osteoporosis occurs when an underlying disease (genetic: cystic fibrosis; endocrine: diabetes, hypogonadism, hyperthyroidism, hyperparathyroidism, Cushing disease; autoimmune: rheumatoid arthritis; etc.), lifestyle behavior (smoking, alcohol abuse, sedentary life) and/or drugs use (glucocorticoids, hypogonadism-inducing agents etc.) causes the excessive bone loss. This type of osteoporosis is mainly common in younger adults and the majority of men with osteoporosis. Approximately one-third of postmenopausal women, and one-half of premenopausal women and men are found to have secondary osteoporosis.

At any rate, the hallmark of osteoporosis is fragility fracture-defined as a fracture happening due to falls from a standing height in response to mechanical forces that would not normally result in fracture. (Cummings S R, Melton L J. Epidemiology and outcomes of osteoporotic fractures. Lancet (London, England). 2002; 359(9319):1761-7. Warriner A H, Patkar N M, Yun H, Delzell E. Minor, major, low-trauma, and high-trauma fractures: what are the subsequent fracture risks and how do they vary? Current osteoporosis reports. 2011; 9(3): 122-8.) Hip, spine, humerus and forearm are the most common skeletal sites where fragility fractures happen and are referred to as major osteoporotic fractures. It is estimated that after the age of 50 years, one in two women and one in four men will suffer a major osteoporotic fracture in their remaining lifetime. (Kanis J A, Johnell O, Oden A, Sembo I, Redlund-Johnell I, Dawson A, et al. Long-term risk of osteoporotic fracture in Malmö. Osteoporosis international: a journal established as result of cooperation between the European Foundation for Osteoporosis and the National Osteoporosis Foundation of the USA. 2000; 11 (8): 669-74.) In women over 45 years of age, osteoporosis accounts for more days spent in hospital than many other diseases, including diabetes, myocardial infarction, and breast cancer. Moreover, a prior fracture is associated with an 86% increased risk of a subsequent fracture. Fractures are associated with high morbidity and mortality; and are often a precursor of disability, loss of independence, and premature death among the elderly. (Binkley N, Blank R D, Leslie W D, Lewiecki E M, Eisman J A, Bilezikian J P. Osteoporosis in Crisis: It's Time to Focus on Fracture. Journal of bone and mineral research: the official journal of the American Society for Bone and Mineral Research. 2017; 32 (7): 1391-4.)

Once the fracture risk is identified, prevention steps are taken on a certain ‘hierarchical’ level: general lifestyle advice; adequate calcium and/or vitamin D supplementation intake in addition to a healthy lifestyle; and/or pharmacological therapy. Based on the mechanism of function, there are two types of anti-osteoporotic pharmacological therapies: antiresorptive agents (such as bisphosphonates, estrogen agonists/antagonists, estrogens, calcitonin and denosumab) which reduce bone resorption; and anabolic agents (such as teriparatide) which stimulate bone formation. More recently, romosozumab has been approved for its bone forming effects. (Tu K N, Lie J D, Wan C K V, Cameron M, Austel A G, Nguyen J K, et al. Osteoporosis: A Review of Treatment Options. P & T: a peer-reviewed journal for formulary management. 2018; 43 (2): 92-104.)

Evidently, fracture—this highly traumatic experience—and its management, come at great social, economic and health burden which would be reinforced by the aging of the population as well as the longer life expectancy. It represents a major challenge for the public health and necessitates particular research focus. As independency is one of the major factors defining the healthy ageing of the population, preventing the loss of independency by preventing fractures is of great importance. The overall management and prevention of osteoporosis requires an accurate clinical assessment (direct or indirect) of bone strength, bone resilience and fracture risk.

In clinical practice, Bone mineral density (BMD), measured by Dual X-ray Absorptiometry (DXA), has been the gold standard for osteoporosis diagnosis in the absence of established fragility fractures. BMD is one of the major determinants of bone strength and fracture risk, but yet considerable overlap (up to 40%) exists in BMD values between individuals who develop fractures and those who do not. Intrinsically, it is accepted that defining osteoporosis on the sole basis of projected bone mineral density (BMD by DXA) has reached its limit. Indeed, the multi-factorial nature of this disease encourages the current definition of osteoporosis to evolve towards a complex risk model based upon clinical risk factors (CRF) and BMD (e.g. FRAX™). Considering CRFs along with BMD in the assessment of fracture risk increases the sensitivity of screening without sacrificing specificity. However, while some of the limitations of the current use of DXA are addressed via the concomitant use of CRFs, it only partially considers information on bone micro-architecture. As such, any additional information on micro-architecture should help to reduce the significant overlap that exists between those with and without a truly increased risk of fracture. However ideally such information should be available in clinical practice and without significantly disrupting the clinical workflow and without adding ionizing radiation to the patient.

EP 1 576 526 describes a process for determining the mechanical strength of a bone from a two-dimensional image of the bone. This process does not depend on BMD or does not depend only on BMD. This process allows to measure a parameter a, also known as Trabecular Bone Score (TBS).

The goal of the invention is to present a process or device allowing to analyze a bone, that does not depend on BMD or does not depend only on BMD, and:
improving the ability to differentiate or predict a bone fracture, and/or
having better measurement reproducibility, and/or
having a better correlation with the micro architecture of the bone, and/or
being less influenced by the physiological characteristics of the patient, for example the volume and/or nature of the soft tissues surrounding the bone, and/or
being less influenced by the choice of the technical parameters of the image acquisition.

### SUMMARY

An aspect of the invention concerns a (preferably computer implemented) process or method for analyzing a texture of a human or animal tissue from a digitized image (preferably obtained by X-Ray based imaging system) comprising a step of calculating at least one texture score B for the image by applying an experimental variogram to the tissue texture, typically to the gray levels of the digitized image.

The process or method according to the invention can comprise a step of applying robustness improvement step(s) that take into account, into the texture score B:
at least one patient factors related to the patient imaged on the image and/or
at least one technical factor related to the acquisition of the image.

The at least one patient factor can comprise:
effect of patient morphology including at least one among:
   effect of soft tissue, and/or tissue thickness, and/or its distribution and/or its composition in the patient, and/or indirect surrogates and/or
   a weight and/or Body Mass Index (BMI) of the patient, and/or
   a size of the patient, and/or
effect of at least one pathology or condition of the patient, and/or
effect of patient positioning during the acquisition of the image.

The at least one technical factor can comprise:
potential defective detectors and sensors for acquiring the image, and/or
effect of scan mode and settings for acquiring the image, and/or
technical characteristics of the imaging device which is used for acquiring the image
effect of the variability in between imaging systems for acquiring the image, and/or
the Signal-Noise-Ratio (SNR) of the image, and/or
the resolution of the image.
The tissue can be a human tissue.

The tissue can be a bone tissue (and preferably a human tissue), and the texture score B is preferably a bone texture score B. In a less preferred variant, the tissue is a soft tissue.

The digitized bi-dimensional image can be chosen in a region having a trabecular structure.

The process or method according to the invention can comprise:
determining an optimized pixel sampling S of the image for which each pixel Pᵢ=(xᵢ,yᵢ)ϵS having its gray level value h(Pᵢ)
for at least one region of interest (ROI) of the pixel sampling S, choosing a predetermined set of directions I depending on the given region of interest (ROI); in a variant, the predetermined set of direction is not chosen depending on the ROI, but is a default predetermined set of directions I;
for each pixel: computing at least one variogram of the gray levels of the sampling S by moving from this pixel by at least one distance rϵ[1, R₀] along those directions I, a variogram being computed for each predetermined direction or for all the predetermined directions at the same time,
for each variogram of each pixel and/or for a global variogram of the sampling S combining the variograms for each pixel, evaluating parameters from the variogram, preferably evaluating at least one of the following parameters on a log-log scale:
   the initial slope a,
   the sill b, representing the asymptote value of the variogram.
   the range c, representing the distance at which the variogram curve transitions from a quasi-linear progression to an asymptotic behavior
   the nugget d, representing the initial value of the variogram, and
   the area under the variogram curve e,
combining:
   the evaluated parameter(s) (preferably a, b, c, d and/or e) obtained for each pixel into a texture score B for each pixel, and/or
   the evaluated parameter(s) (preferably a, b, c, d and/or e) obtained for sampling S into a texture score B for the sampling S, and/or
   the evaluated parameter(s) (preferably a, b, c, d and/or e) obtained for each pixel into a texture score B for the sampling S
The combining step can further comprise applying the robustness improvement step related to the at least one patient factor and/or the at least one technical factor into the texture score B, preferably:
   by determining and/or correcting, as a function of patient and/or technical factor(s), and before or during the determination or calculation of score B, at least one parameter used for calculating or determining score B, preferably:
      at least one parameter among R₀, a, b, c, d, and/or
      at least one parameter (α,β,γ,δ,ε) used for giving respective weights between a, b, c, d, and/or e for calculating the texture score B and/or
by correcting, as a function of patient and/or technical factor(s), score B.

The predetermined set of directions I can depend on:
a skeletal site of a bone, the human or animal tissue being the bone, and/or
the region of interest (ROI), and/or
a resolution of the image, and/or
a signal/noise ratio of the image.

The step of choosing predetermined set of directions I can be done by determining:
a set of N directional vectors U={\vec{u_{θ}_{₁}}, . . . , {right arrow over (u_{θ}_{_{N}})}}, where θₖϵ[−π,π], ∀kϵS 1, . . . , N.

Moving by a distance r can be done by, for each pixel Pᵢ=(xᵢ,yᵢ)ϵS and each direction \vec{u_{θ}_{₁}}ϵU, moving along \vec{u_{θ}_{₁}} to a distance rϵ[1, R₀] in pixels, h(Pᵢ+r*\vec{u_{θ}_{₁}}) being the gray value of such pixel.

A variogram can be computed for all the predetermined directions at the same time, the step of computing the variogram of the gray levels as a function of the distance r being preferably done by averaging the squared differences of h over several pairs of pixels, each at distance r preferably with the formula:

$${V}_{{P}_{i}} ( r ) = \frac{1}{N} * {\sum }_{k = 1}^{k = N} {[ h ( {P}_{i} ) - h ( {P}_{i} + {r}^{*} \vec{{u}_{{θ}_{k}}} ) ]}^{2} ,$$

V_{P}_{ᵢ}(r) being computed for every pixel PᵢϵS,
   A variogram can be computed for each predetermined direction, h(O) being the gray level of an initial given pixel before moving, h(r) being the gray level of a given new pixel after moving by a distance r along one of the predetermined directions from the initial given pixel, the variogram being preferably computed with the formula: Vᵢ(r)=[h(r)−h(O)]² where i∈I

Each evaluated parameter (preferably a, b, c, d and/or e) can be evaluated as a least squares regression model of the considered variogram.

The evaluated parameter(s) (preferably a, b, c, d and/or e) can be combined into the texture score B using linear or nonlinear equations, preferably depending on a clinical context.

The process or method according to the invention can comprise for each variogram of each pixel and/or for a global variogram of the sampling S combining the variograms for each pixel, evaluating at least one of the following parameters on a log-log scale:
the sill b, representing the asymptote value of the variogram
the range c, representing the distance at which the variogram curve transitions from a quasi-linear progression to an asymptotic behavior
the nugget d, representing the initial value of the variogram, and
the area under the variogram curve e.

The process or method according to the invention can comprise for each variogram of each pixel and/or for a global variogram of the sampling S combining the variograms for each pixel, evaluating at least two of the following parameters on a log-log scale:
the initial slope a,
the sill b, representing the asymptote value of the variogram
the range c, representing the distance at which the variogram curve transitions from a quasi-linear progression to an asymptotic behavior
the nugget d, representing the initial value of the variogram, and
the area under the variogram curve e.

The process or method according to the invention can comprise for each variogram of each pixel and/or for a global variogram of the sampling S combining the variograms for each pixel, evaluating the initial slope a and at least one of the following parameters on a log-log scale:
the sill b, representing the asymptote value of the variogram
the range c, representing the distance at which the variogram curve transitions from a quasi-linear progression to an asymptotic behavior
the nugget d, representing the initial value of the variogram, and
the area under the variogram curve e.

The texture score B is preferably unitless.

An other aspect of the invention concerns a computer program comprising instructions which, when executed by a computer, implement the steps of the process or method according to the invention.

An other aspect of the invention concerns a computer program product comprising instructions which, when the program is executed by a computer, cause the computer to carry out the steps of the process or method according to the invention.

An other aspect of the invention concerns a computer-readable storage medium comprising instructions which, when executed by a computer, cause the computer to carry out the steps of the process or method according to the invention.

Another aspect of the invention concerns a device for analyzing a texture of a human or animal tissue from a digitized image (preferably obtained by X-Ray based imaging system) comprising means arranged to and/or programmed to calculate at least one texture score B for the image by applying an experimental variogram to the tissue texture, typically to the gray levels of the digitized image.

The device according to the invention can further comprise means arranged to and/or programmed to apply robustness improvement step that take into account, into the texture score B:
at least one patient factor related to the patient imaged on the image and/or
at least one technical factor related to the acquisition of the image.

The at least one patient factor can comprise:
effect of patient morphology including at least one among:
   effect of soft tissue, and/or tissue thickness, and/or its distribution and/or its composition in the patient, and/or indirect surrogates and/or
   a weight and/or Body Mass Index (BMI) of the patient, and/or
   a size of the patient, and/or
effect of at least one pathology or condition of the patient, and/or
effect of patient positioning during the acquisition of the image.

The at least one technical factor can comprise:
potential defective detectors and sensors for acquiring the image, and/or
effect of scan mode and settings for acquiring the image, and/or
technical characteristics of the imaging device which is used for acquiring the image
effect of the variability in between imaging systems for acquiring the image, and/or
the Signal-Noise-Ratio (SNR) of the image, and/or
the resolution of the image.

The tissue can be a human tissue.

The tissue can be a bone tissue (and preferably a human tissue), and the texture score B is preferably a bone texture score B. In a less preferred variant, the tissue is a soft tissue.

The digitized bi-dimensional image can be an image imaging a trabecular structure.

The device according to the invention can comprise:
means arranged to and/or programmed to determine an optimized pixel sampling S of the image for which each pixel Pᵢ=(xᵢ,yᵢ)ϵS having its gray level value h(Pᵢ)
for at least one region of interest (ROI) of the pixel sampling S, means arranged to and/or programmed to choose a predetermined set of directions I depending on the given region of interest (ROI); the means are optional, because in a variant, the predetermined set of direction is not chosen depending on the ROI, but is a default predetermined set of directions I;
for each pixel: means arranged to and/or programmed to compute at least one variogram of the gray levels of the sampling S by moving from this pixel by at least one distance rϵ[1, R₀] along those directions I, and arranged to and/or programmed to compute a variogram for each predetermined direction or for all the predetermined directions at the same time,
for each variogram of each pixel and/or for a global variogram of the sampling S combining the variograms for each pixel, means arranged to and/or programmed to evaluate parameters from the variogram, preferably to evaluate at least one of the following parameters on a log-log scale:
   the initial slope a,
   the sill b, representing the asymptote value of the variogram.
   the range c, representing the distance at which the variogram curve transitions from a quasi-linear progression to an asymptotic behavior
   the nugget d, representing the initial value of the variogram, and
   the area under the variogram curve e,
means arranged to and/or programmed to combine:
   the evaluated parameter(s) (preferably a, b, c, d and/or e) obtained for each pixel into a texture score B for each pixel, and/or
   the evaluated parameter(s) (preferably a, b, c, d and/or e) obtained for sampling S into a texture score B for the sampling S, and/or
   the evaluated parameter(s) (preferably a, b, c, d and/or e) obtained for each pixel into a texture score B for the sampling S

The device according to the invention can further comprise means arranged to and/or programmed to apply the robustness improvement step related to the at least one patient factor and/or the at least one technical factor into the texture score B, preferably:
by determining and/or correcting, as a function of patient and/or technical factor(s), and before or during the determination or calculation of score B, at least one parameter used for calculating or determining score B, preferably:
   at least one parameter among R₀, a, b, c, d, and/or
   at least one parameter (α,β,γ,δ,ε) used for giving respective weights between a, b, c, d, and/or e for calculating the texture score B
      and/or
by correcting, as a function of patient and/or technical factor(s), score B.

The predetermined set of directions I can depend on:
a skeletal site of a bone, the human or animal tissue being the bone, and/or
the region of interest (ROI), and/or
a resolution of the image, and/or
a signal/noise ratio of the image.

The means arranged to and/or programmed to choose predetermined set of directions I can be arranged to and/or programmed to choose predetermined set of directions I by determining:
a set of N directional vectors U={\vec{u_{θ}_{₁}}, . . . , {right arrow over (u_{θ}_{_{N}})}}, where θₖϵ[−π,π], ∀k∈1, . . . , N.

The means arranged to and/or programmed to compute at least one variogram can be arranged to and/or programmed to compute at least one variogram of the gray levels of the sampling S by moving by a distance r, for each pixel Pᵢ=(xᵢ,yᵢ)ϵS and each direction \vec{u_{θ}_{ₖ}}ϵU, along \vec{u_{θ}_{ₖ}} to a distance rϵ[1, R₀] in pixels, h(Pᵢ+\vec{u_{θ}_{ₖ}}) being the gray value of such pixel.

The means arranged to and/or programmed to compute at least one variogram can be arranged to and/or programmed to compute a variogram for all the predetermined directions at the same time, the means arranged to and/or programmed to compute at least one variogram being preferably arranged to and/or programmed to compute the variogram of the gray levels as a function of the distance r by averaging the squared differences of h over several pairs of pixels, each at distance r preferably with the formula:

$${V}_{{P}_{i}} ( r ) = \frac{1}{N} * {\sum }_{k = 1}^{k = N} {[ h ( {P}_{i} ) - h ( {P}_{i} + {r}^{*} \vec{{u}_{{θ}_{k}}} ) ]}^{2} ,$$

V_{P}_{ᵢ}(r) being computed for every pixel Pᵢ∈S,

The means arranged to and/or programmed to compute at least one variogram can be arranged to and/or programmed to compute a variogram for each predetermined direction, h(O) being the gray level of an initial given pixel before moving, h(r) being the gray level of a given new pixel after moving by a distance r along one of the predetermined directions from the initial given pixel, the means arranged to and/or programmed to compute at least one variogram being preferably arranged to and/or programmed to compute the variogram preferably with the formula: Vᵢ(r)=[h(r)−h(O)]² where i∈I

The means arranged to and/or programmed to evaluate the parameters can be arranged to and/or programmed to evaluate each parameter (preferably a, b, c, d, and/or e) as a least squares regression model of the considered variogram.

The means arranged to and/or programmed to combine the parameters can be arranged to and/or programmed to combine the evaluated parameter(s) (preferably a, b, c, d and/or e) into the texture score B using linear or nonlinear equations, preferably depending on a clinical context.

The means arranged to and/or programmed to evaluate the parameters can be arranged and/or programmed, for each variogram of each pixel and/or for a global variogram of the sampling S combining the variograms for each pixel, to evaluate at least one of the following parameters on a log-log scale:
the sill b, representing the asymptote value of the variogram
the range c, representing the distance at which the variogram curve transitions from a quasi-linear progression to an asymptotic behavior
the nugget d, representing the initial value of the variogram, and
the area under the variogram curve e.

The means arranged to and/or programmed to evaluate the parameters can be arranged and/or programmed, for each variogram of each pixel and/or for a global variogram of the sampling S combining the variograms for each pixel, to evaluate at least two of the following parameters on a log-log scale:
the initial slope a,
the sill b, representing the asymptote value of the variogram
the range c, representing the distance at which the variogram curve transitions from a quasi-linear progression to an asymptotic behavior
the nugget d, representing the initial value of the variogram, and
the area under the variogram curve e.

The means arranged to and/or programmed to evaluate the parameters can be arranged and/or programmed, for each variogram of each pixel and/or for a global variogram of the sampling S combining the variograms for each pixel, to evaluate the initial slope a and at least one of the following parameters on a log-log scale:
the sill b, representing the asymptote value of the variogram
the range c, representing the distance at which the variogram curve transitions from a quasi-linear progression to an asymptotic behavior
the nugget d, representing the initial value of the variogram, and
the area under the variogram curve e.

The texture score B is preferably unitless.

### BRIEF DESCRIPTION OF THE DRAWINGS

Other advantages and characteristics of the invention will appear upon examination of the detailed description of embodiments which are in no way limitative, and of the appended drawings in which:

FIG. 1 illustrates a reconstructed image from a DXA spine scan,

FIG. 2 illustrates a sampling region, corresponding to the measurement area in an embodiment of the process according to the invention which is a best realization mode,

FIG. 3 illustrates a sub-sampling region of the bone site, corresponding to the measurement area in a variant according to the invention,

FIG. 4 a reconstructed image from a DXA spine scan, with the determination of a set of directions I in the embodiment of the process according to the invention,

FIG. 5 illustrates an example of variogram V and its least squares regression curve with computation range R₀ of 20 mm in the embodiment of the process according to the invention,

FIG. 6 illustrates a curve V (corresponding to FIG. 5) on log-log scale in the embodiment of the process according to the invention,

FIG. 7 the curve V on log-log scale in the embodiment of the process according to the invention, with the parameters a, b, c, d and e

FIG. 8 illustrates the constitution and/or the correlation of bone Strength and Bone Texture,

FIG. 9 illustrates an age, ethnicity, gender control curve in the embodiment of the process according to the invention,

FIG. 10 is a table combining bone Texture Score B with Bone Density Status or BMD in the embodiment of the process according to the invention, in order to obtain a Bone Health category (1 to 9) corresponding to a Fracture Risk,

FIG. 11 illustrates the Fracture Risk Assessment Tool (FRAX™) adjusted by Texture Score B in the embodiment of the process according to the invention,

FIG. 12 block diagram representing the steps a) to i) of the embodiment of the process according to the invention; FIG. 12 is separated in FIG. 12A and FIG. 12B.

### DETAILED DESCRIPTION

These embodiments being in no way limitative, we can consider variants of the invention including only a selection of characteristics subsequently described or illustrated, isolated from other described or illustrated characteristics (even if this selection is taken from a sentence containing these other characteristics), if this selection of characteristics is sufficient to give a technical advantage or to distinguish the invention over the state of the art. This selection includes at least one characteristic, preferably a functional characteristic without structural details, or with only a part of the structural details if that part is sufficient to give a technical advantage or to distinguish the invention over the state of the art.

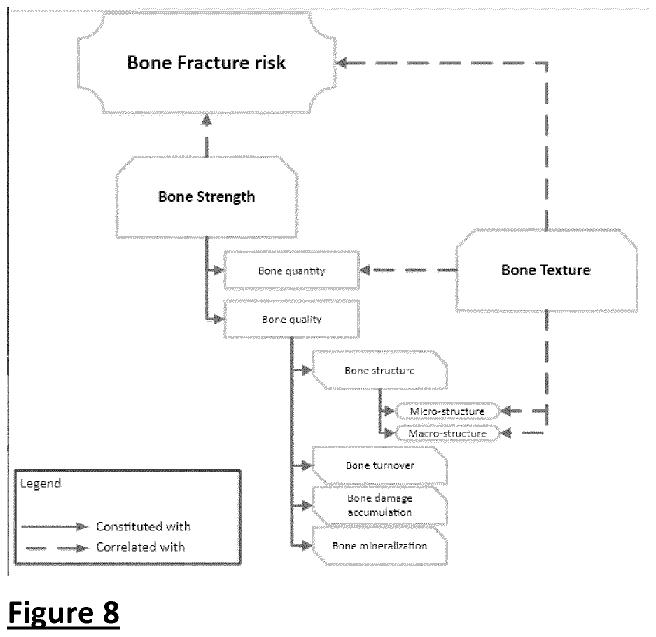

In this specification, we will use the following definition (see FIG. 8):

Bone strength: is defined as the ultimate stress which the bone structure can bear before failure, which is the maximum stress in the stress-strain curve. Bone strength's determinants are multiple and include for example several anatomical properties such as the mineral density and the micro and macro-architecture and geometry. It comprises Bone quantity and Bone quality.

Bone quantity i.e. bone density: Corresponds to the total amount of calcified tissue in the bone. Evaluated by the bone density, in grams per unit of volume or grams per unit of surface. Bone density can be evaluated by devices like DXA densitometers; Quantitative computed tomography (QCT), peripheral Quantitative computed tomography (pQCT) or High-Resolution peripheral Quantitative computed tomography (HR-pQCT) scanners; or specialized ultrasound devices.

Bone quality: Corresponds to the global qualitative state of the bone. According to the NIH definition, it is comprised of bone turnover, bone mineralization, bone damage accumulation and bone architecture or bone structure. The “bone architecture” or “bone structure” can be divided in macro-architecture and micro-architecture.

Macro-architecture (also known as macro-structure): Corresponds mainly to the overall geometry of the bone, its shape and its distribution. It also includes the differentiation into cortical bone and cancellous bone (also referred to as trabecular bone).

Micro-architecture (also known as micro-structure): Corresponds to the internal structure of the trabecular bone and can be described by a combination of parameters such as trabecular thickness and separation of trabeculae, trabecular number, trabecular connectivity, and parameters comprehensively describing the network architecture, such as the structure model index (SMI).

Bone mineralization: Corresponds to the quantity and intrinsic quality of the organic matrix of collagen and hydroxy-apatite crystals.

Bone turnover: Bone turnover includes the processes of bone resorption and bone formation. Bone turnover can be indirectly evaluated by the measurement of biochemical markers (blood and/or urine) and by analysis of biopsy samples (from histomorphometry for example).

Bone damage accumulation: it is corresponding to the gradual gathering of damages acquired on the bone

Bone texture: statistical properties of the bone, it can be linked to the micro and macro architecture of the bone.

Bone resilience index: it is an index of the resistance to fracture based on the combination of both bone mineral density value or bone mineral density T-score and texture score.

Bone fracture risk: it is addressing a situation and the medical conditions which are involving exposure to break in the continuity of a bone, a fracture. It is linked to the bone strength and bone resilience.

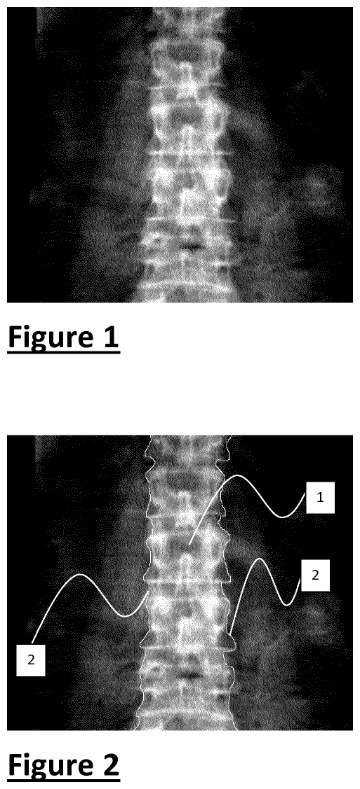

We are now going to describe, in reference to FIGS. 1 to 12, an embodiment of a process according to the invention implemented by a device according to the invention.

Based on the prior art process described in the patent references (CA2549281A1, EP1576526B1, FR_2848694, US_7609867), the analysis of the two-dimensional medical x-ray-based images has been enhanced according to the invention to determine a bone texture score B, related to some aspects of bone quality e.g., bone macro and micro architecture.

The bone texture score B can predict osteoporotic fracture for both males and females independently of bone mineral density and most common clinical risk factors.

The bone texture score B in this embodiment of the invention is interpreted in regard to both age and gender matched controls, and the patient's fragility fracture risk (its position with respect different status of the bone microarchitecture based on tertiles: high bone texture score B compatible with Normal microarchitecture and lower risk of fragility fracture, and low bone texture score B compatible with Degraded microarchitecture and higher risk of fragility fracture).

This embodiment of a process according to the invention is a process for analyzing a texture of a human or animal tissue from a digitized image, preferably obtained by X-Ray based imaging system, comprising a step of calculating at least one texture score B for the image by applying an experimental variogram to the tissue texture (typically to the gray levels) of the digitized image. As described below in step i), this embodiment of a process according to the invention preferably comprises applying robustness improvement step(s) that take into account, into the texture score B (preferably that take into account, into the calculation of the texture score B):
at least one patient factor related to the patient imaged on the image and/or
at least one technical factor related to the acquisition of the image.

By “variogram” (without specifying that it is a theoretical or experimental or empirical one), it is meant in the present description an “experimental variogram” (also called by the skilled in the art “Empirical variogram”).

In statistics the theoretical variogram is a function describing the degree of spatial dependence of a spatial random field or stochastic process.

A theoretical variogram is the average squared difference between the values at two points (in space, in an image, etc.) separated at a distance.

The theoretical variogram can be defined, equivalently, as the variance of the difference between field values at two locations.

The experimental variogram is an estimator of the theoretical variogram from the data. The experimental variogram is used for measured data where the sample information is not available for every location. For instance the variations of intensity of pixels in a radiologic digitized image acquired by a X-Ray instrument sensor, where each pixel Pᵢ=(xᵢ,yᵢ)ϵS is having its gray level value h(Pᵢ).

The notion of variogram is clear to the one skilled in the art, see for example https://fr.wikipedia.org/wiki/Variogramme and https://en.wikipedia.org/wiki/Variogram.

See also:
Matheron, Georges (1963). “Principles of geostatistics”. Economic Geology. 58 (8): 1246-1266. doi: 10.2113/gsecongeo.58.8.1246. ISSN 1554-0774.
Ford, David. “The Empirical Variogram”
Bachmaier, Martin; Backes, Matthias (2008-02-24). “Variogram or semivariogram? Understanding the variances in a variogram”. Precision Agriculture. Springer Science and Business Media LLC. 9 (3): 173-175. doi: 10.1007/s11119-008-9056-2. ISSN 1385-2256.
Nguyen, H.; Osterman, G.; Wunch, D.; O'Dell, C.; Mandrake, L.; Wennberg, P.; Fisher, B.; Castano, R. (2014). “A method for colocating satellite XCO2 data to ground-based data and its application to ACOS-GOSAT and TCCON”. Atmospheric Measurement Techniques. 7 (8): 2631-2644. Bibcode: 2014AMT . . . 7.2631N. doi: 10.5194/amt-7-2631-2014. ISSN 1867-8548.
Arregui Mena, J. D.; et al. (2018). “Characterisation of the spatial variability of material properties of Gilsocarbon and NBG-18 using random fields”. Journal of Nuclear Materials. 511:91-108. Bibcode: 2018JNuM . . . 511 . . . 91A. doi: 10.1016/j.jnucmat.2018.09.008.
Schiappapietra, Erika; Douglas, John (April 2020). “Modelling the spatial correlation of earthquake ground motion: Insights from the literature, data from the 2016-2017 Central Italy earthquake sequence and ground-motion simulations”. Earth-Science Reviews. 203:103139. Bibcode: 2020ESRv . . . 20303139S. doi: 10.1016/j.earscirev.2020.103139.
Sokolov, Vladimir; Wenzel, Friedemann (2011-07-25). “Influence of spatial correlation of strong ground motion on uncertainty in earthquake loss estimation”. Earthquake Engineering & Structural Dynamics. 40 (9): 993-1009. doi: 10.1002/eqe.1074.
Olea, Ricardo A. (1991). Geostatistical Glossary and Multilingual Dictionary. Oxford University Press. pp. 47, 67, 81. ISBN 9780195066890.
Cressie, N., 1993, Statistics for spatial data, Wiley Interscience.
Chiles, J. P., P. Delfiner, 1999, Geostatistics, Modelling Spatial Uncertainty, Wiley-Interscience.
Wackernagel, H., 2003, Multivariate Geostatistics, Springer.
Burrough, P. A. and McDonnell, R. A., 1998, Principles of Geographical Information Systems.
Isobel Clark, 1979, Practical Geostatistics, Applied Science Publishers.
Clark, I., 1979, Practical Geostatistics, Applied Science Publishers.
David, M., 1978, Geostatistical Ore Reserve Estimation, Elsevier Publishing.
Hald, A., 1952, Statistical Theory with Engineering Applications, John Wiley & Sons, New York.
Journel, A. G. and Huijbregts, Ch. J., 1978 Mining Geostatistics, Academic Press.

The texture score B is unitless.

The embodiment of a process according to the invention will now be described in details.

This embodiment of a process according to the invention comprises the step of acquiring a digitized image, preferably obtained by X-Ray based imaging system.

An example of acquired 2D image is illustrated in FIG. 1. FIG. 1 illustrates a reconstructed image from a DXA spine scan.

This digitized image can be a bi-dimensional image or a projected three-dimensional image.

From the x-ray imaging modalities (such as, but not restricted to digital x-ray, projected Computed Tomography (CT), projected QCT, DXA, conventional x-ray imaging), the available images are not always suited to texture analysis. To improve the contents of the input image for our process, we reconstruct an image using, as much as possible, unprocessed data. This unprocessed data includes for example raw detector data, scan parameters and analysis data.

The 2D x-ray-based image is used as an input for our computation. This image is reconstructed from data retrieved from an imaging system. This data is including but not restricted to sensor data, scan parameters, scan analysis data.

The human tissue is a bone tissue, but preferably a bone tissue, and the texture score B is preferably a bone texture score B (in a variant of this embodiment, the tissue could be an animal tissue and/or the tissue could be a soft tissue).

In this example embodiment, the digitized bi-dimensional image is chosen in a region having a trabecular bone structure.

On such reconstructed image, the computation process steps are the following steps a) to i) illustrated in FIG. 12 and j):
a) determining an optimized pixel sampling S of the image for which each pixel Pᵢ=(xᵢ,yᵢ)ϵS has (or is defined by) its gray level value h(Pᵢ).

As illustrated in FIG. 2, from the reconstructed input image, a pixel sampling S is determined to select the locations on which the variogram Vᵢ is evaluated (in step d)).

In FIG. 2, sampling S is referenced 1 and is delimited between the lines 2.

Those locations are optimized so that the final bone texture score B is adapted and efficient in a given clinical context and anatomical site. In the most basic sense, the sampling region corresponds to the region in which the bone is present in the image.

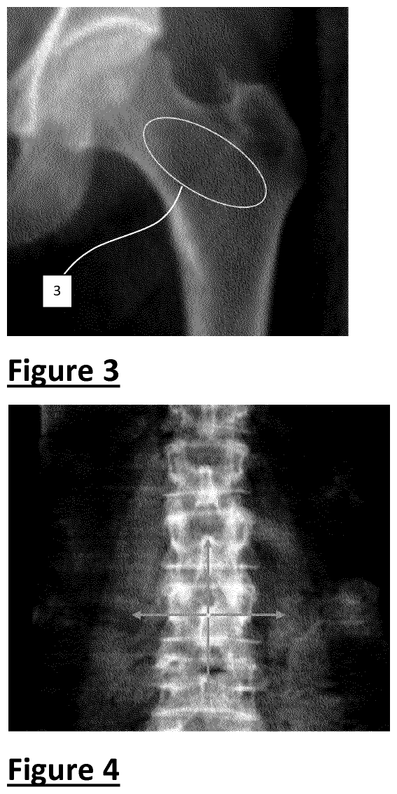

Depending on the bone site, a sub-sample 3 (see FIG. 3) of this region in which the bone is present in the image may be used to increase the performance.
b) for at least one region of interest (ROI) of the pixel sampling S, preferably for a plurality of ROI:
   determining a computation distance R₀ depending on the given region of interest (ROI); The range R₀ is determined depending on the bone skeletal site and the image resolution. On DXA systems, the value R₀ is between 1 cm and 2 cm.
   choosing a predetermined set of directions I, depending on the given region of interest (ROI). This step of choosing predetermined set of directions I is done by determining a set of N directional unit vectors U={\vec{u_{θ}_{₁}}, . . . , {right arrow over (u_{θ}_{_{N}})}} (N being a positive integer number), where θₖϵ[−π,π], ∀k∈1, . . . , N, θₖ being the angle, around a considered pixel, carrying the vector \vec{u_{θ}_{ₖ}}.

Typically N>2.

The maximum value of N depends on the complexity of the bone structure of the considered imaged bone of the ROI and its image resolution. Typically N<9 for a bone having a non-complex bone structure such like vertebra or lumbar spine, but in some cases N can increase significantly for complex structures such as the proximal femur.

For example: N=3 or 4 or 6 or 8.

The N directional vectors are preferably distributed uniformly at an angle 2π/N around the considered pixel.

The predetermined set of directions I (vectors U={\vec{u_{θ}_{₁}}, . . . , {right arrow over (u_{θ}_{_{N}})}}) depends on:
a skeletal site of a bone on the image and/or on the ROI, the human or animal tissue being the bone, and/or
the considered region of interest (ROI), and/or
a resolution of the image, and/or
a signal/noise ratio of the image.

This determination of a set of directions I:
improves the ability to differentiate or predict a bone fracture, and/or
allows a better measurement reproducibility (or precision), and/or
allows a better correlation with the micro architecture of the bone.

Thus, to compute the variogram V_{P}_{ᵢ} in later step d), the pixel with value h(Pᵢ) is compared to pixels located along lines with specific directions.

Those directions depend both on the type of bone (i.e. skeletal site) and the region of interest selected for measurement on this bone to optimize texture measurements.

Indeed, the selected direction(s) are linked to the morphology of the bone, especially the direction of the trabeculae of the cancellous bone. For example at the spine, the preferred direction of the trabeculae is vertical, so the selected directions will be vertical and horizontal [−π/2, 0, π/2, π] (parallel and perpendicular to the orientation of the trabeculae) as illustrated by the four arrows in FIG. 4.
c) for each pixel Pᵢ=(xᵢ,yᵢ)ϵS and each direction \vec{u_{θ}_{ₖ}}ϵU, moving along \vec{u_{θ}_{ₖ}} to a distance rϵ[1, R₀] (in pixels). We note h(Pᵢ+r*\vec{u_{θ}_{ₖ}}) the gray value of such pixel,
d) for each pixel: computing at least one variogram of the gray levels of the sampling S as a function of the distance r along those directions I (i.e. by moving from this pixel by at least one distance rϵ[1, R₀]; as explained previously, moving by a distance r is done by, for each pixel Pᵢ=(xᵢ,yᵢ)ϵS and each direction \vec{u_{θ}_{ₖ}}ϵU, moving along \vec{u_{θ}_{ₖ}} to a distance rϵ[1, R₀] in pixels, h(Pᵢ+r*\vec{u_{θ}_{ₖ}}) being the gray value of such pixel), a variogram being computed:
   for each predetermined direction one by one (i.e. one variogram per pixel in S and per direction among I); if a variogram is computed for each predetermined direction, h(O) being the gray level of an initial given pixel before moving, h(r) being the gray level of a given new pixel after moving by a distance r along one of the predetermined directions from the initial given pixel, the variogram being computed with the formula: Vᵢ(r)=[h(r)−h(O)]² where i∈I; or
   for all the predetermined directions at the same time (i.e. one variogram per pixel in S); If a variogram is computed for all the predetermined directions at the same time, the step of computing the variogram of the gray levels as a function of the distance r is done by averaging the squared differences of h over several pairs of pixels, each at distance r with the formula:

$${V}_{{P}_{i}} ( r ) = \frac{1}{N} * {\sum }_{k = 1}^{k = N} {[ h ( {P}_{i} ) - h ( {P}_{i} + {r}^{*} \vec{{u}_{{θ}_{k}}} ) ]}^{2} ,$$

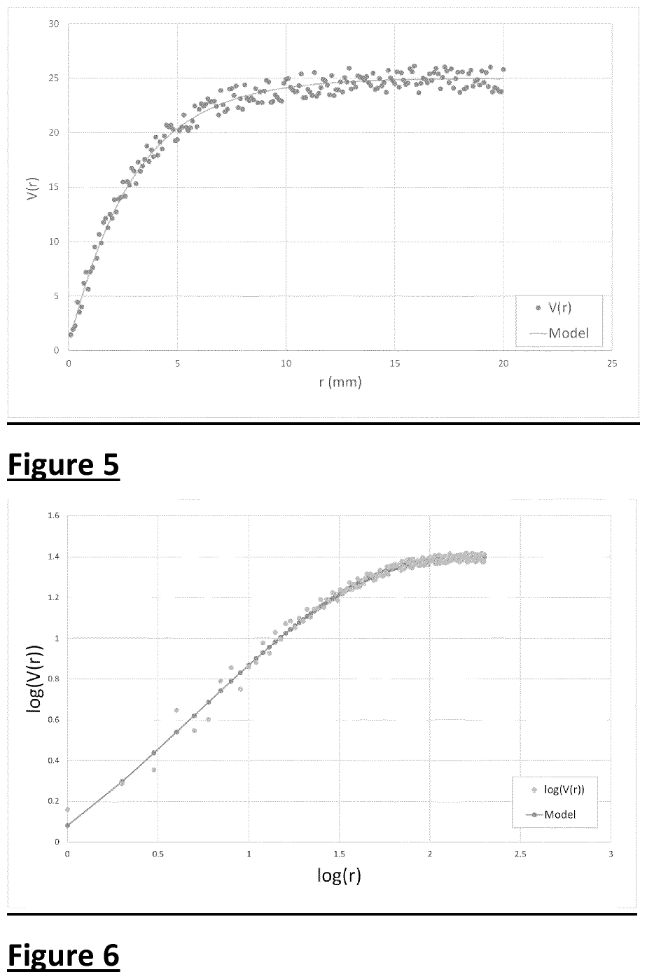

V_{P}_{ᵢ}(r) being computed for every pixel PᵢϵS. An example variogram V(r) for one pixel and its least squares regression curve is given in FIG. 5.

The formula for V_{P}_{ᵢ} is applied to each PᵢϵS for a given range of values rϵ[1, R₀]. This specific range of values for r is selected to allow V_{P}_{ᵢ}:r [symbol] V_{P}_{ᵢ}(r) to converge for the evaluation of all the required parameters of the variogram V_{P}_{ᵢ}.

The range R₀ is determined depending on the bone skeletal site and the image resolution. On DXA systems, the value R₀ is between 1 cm and 2 cm.

The range of computation R₀ is not to be confused with the range parameter c of the variogram model.

e) computing V_{P}_{ᵢ}(r) for every pixel Pᵢ∈S, and tracing or calculating or determining the associated curves on a log-log scale. An example variogram V(r) on a log-log scale for one pixel is given in FIG. 6.

The representation of the variogram curve V_{P}_{ᵢ} in a log-log scale implies that the values along each axis no longer have units.
f) evaluating the full model V as a least squares regression model of V_{P}_{ᵢ} representations,
g) evaluating the parameters of this model including but not limited to the initial slope a, the sill b, the range c, the nugget d, and/or the area under the curve e.

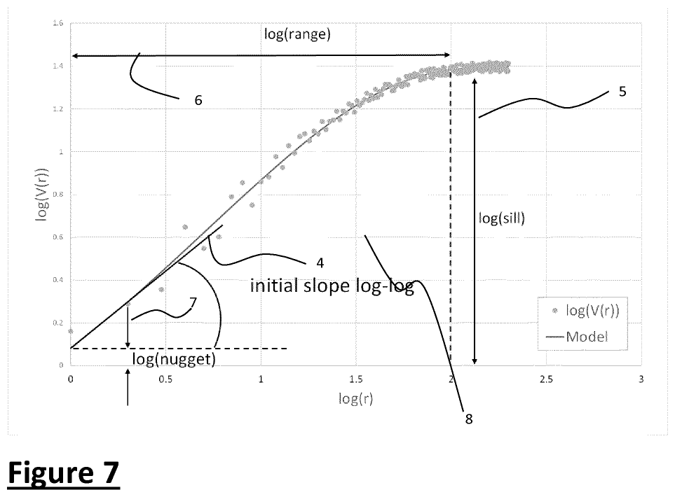

For each variogram V_{P}_{ᵢ} of each pixel and/or for the global variogram V of the sampling S combining the variograms for each pixel, evaluating at least one of the following parameters on a log-log scale:
the initial slope a of the variogram (referenced 4 in FIG. 7),
the sill b, representing the asymptote value of the variogram (referenced 5 in FIG. 7),
the range c, representing the distance at which the variogram curve transitions from a quasi-linear progression to an asymptotic behavior (referenced 6 in FIG. 7),
the nugget d, representing the initial value of the variogram (referenced 7 in FIG. 7), and
the area under the variogram curve e of the variogram (referenced 8 in FIG. 7).

On the log-log plots of V_{P}_{ᵢ} or V, we fit a mathematical model. These parameters (i.e. coefficients) a, b, c, d, e of this model are combined to create the bone texture score B.

Each parameter a, b, c, d, and/or e is evaluated from a least squares regression model of the considered variogram.

Depending on the contents of the image, the selected coefficients of the model may vary, because they may not be clearly defined (for example, the variogram curve may not converge to an asymptote, and thus “range” might not be defined)

Preferably, the process comprises for each variogram of each pixel and/or for a global variogram of the sampling S combining the variograms for each pixel, evaluating at least one of the parameters sill b, range c, the nugget d, area e on a log-log scale.

Preferably, the process comprises for each variogram of each pixel and/or for a global variogram of the sampling S combining the variograms for each pixel, evaluating at least two of the parameters slope a, sill b, range c, the nugget d, area e on a log-log scale.

Preferably, the process comprises for each variogram of each pixel and/or for a global variogram of the sampling S combining the variograms for each pixel, evaluating the initial slope a and at least one of the following parameters sill b, range c, the nugget d, area e on a log-log scale.

Preferably, the process comprises for each variogram of each pixel and/or for a global variogram of the sampling S combining the variograms for each pixel, evaluating all the parameters slope a, sill b, range c, the nugget d, area e on a log-log scale.
h) combining the at least one parameter(s) a, b, c, d and/or e into the bone texture score B (unitless), for example by using linear or nonlinear equations depending on clinical context.

For example, the process can comprise the step of combining:
the at least one evaluated parameter(s) a, b, c, d and/or e obtained for the variogram of a or each pixel into a texture score B for the or each pixel, and/or
the at least one evaluated parameter(s) a, b, c, d and/or e obtained for the global variogram of sampling S into a global texture score B for the sampling S, and/or
the at least one evaluated parameter(s) a, b, c, d and/or e obtained for the variogram of each pixel into a global texture score B for the sampling S

At least two parameters among a, b, c, d, e are preferably combined into the texture score B using linear or nonlinear equations depending on a clinical context. Indeed, the parameters of the variogram model are combined together into the bone texture parameter B, using combination equations. As an example, such combination equations could include but not be restricted to a multiple linear model for a given clinical context and anatomical site. In such case scenario, B would be defined as:

$$B = α a + β b + γ c + δ d + ε e ,$$

where coefficients α, β, γ, δ, ϵ are respectively associated with the slope, the sill, the nugget and the area under the curve. These coefficients are carefully defined beforehand during clinical performance optimization phases.

These coefficients are typically obtained from different experimental analysis.

Selection of the best coefficients is obtained for example on a large set of clinical studies and images, and further to a grid search optimization phase.

More generally α, β, γ, δ, and/or ε can be obtained in different manners including:
corrective abacus based on experimental measures, and/or
mathematical model and/or simulations of correction based on theory, and/or
machine learning or Artificial Intelligence (AI) algorithms, and/or
selection of the best coefficients obtained for example on a large set of clinical studies and images, and further to a grid search optimization phase with clinical performance outcome, and/or
and the combination of the above methods.

This combination of at least two parameters improves the ability to differentiate or predict a bone fracture.
i) applying the robustness improvement step(s) related to the at least one patient factor and/or the at least one technical factor into the texture score B, preferably related to both patient and technical factors.

The robustness step(s) may be implemented:
before or during previous step h) (by determining and/or correcting, as a function of patient and/or technical factor(s), and before or during the determination or calculation of score B, at least one parameter used for calculating or determining score B; preferably by determining and/or correcting R₀, α, β, γ, δ, ε, a, b, c, d, and/or e, as a function of patient and/or technical factor(s), before or during the determination or calculation of score B), and/or
after previous step h) (by correcting, as a function of patient and/or technical factor(s), score B (obtained at least from parameter(s) R₀, α, β, γ, δ, ε, a, b, c, d, and/or e)).

The robustness step(s) may be implemented (by determining and/or correcting R₀, α, β, γ, δ, ε, a, b, c, d, e, and/or B) in different manners including:
corrective abacus based on experimental measures taking into account the patient and/or technical factor(s) and their observed effect on the variogram and/or texture score B, and/or
mathematical model and/or simulations of correction based on theory taking into account the patient and/or technical factor(s) and their predicted effect on the variogram and/or texture score B, and/or
machine learning or Artificial Intelligence (AI) algorithms learning to minimize the effect on the variogram and/or texture score B of the patient and/or technical factor(s), and/or
selection of the best coefficients obtained for example on a large set of clinical studies and images, and further to a grid search optimization phase with clinical performance outcome, up to the point where the patient and/or technical factor(s) have minimized effect on the variogram and/or texture score B, and/or
and the combination of the above methods.

For example, R₀ is determined and/or corrected as a function of the image resolution of the X-Ray acquired image.

The at least one patient factor comprise:
effect of patient morphology including at least one among:
   effect of soft tissue, and/or tissue thickness, and/or its distribution and/or its composition in the patient, and/or indirect surrogates and/or
   a weight and/or Body Mass Index (BMI) and/or belly circumference of the patient, and/or
   a size of the patient, and/or
effect of at least one pathology or condition of the patient (arthrosis, ascites, aortic calcification, gas, etc.), and/or
effect of patient positioning during the acquisition of the image.

The at least one technical factor comprise:
potential defective detectors and sensors for acquiring the image, and/or
effect of scan mode and settings for acquiring the image, and/or
technical characteristics of the imaging device which is used for acquiring the image
effect of the variability in between imaging systems for acquiring the image, and/or
the Signal-Noise-Ratio (SNR) of the image, and/or
the resolution of the image.

The robustness improvement step allows:
to be less influenced by the physiological characteristics of the patient, for example the volume and/or nature of the soft tissues surrounding the bone, and/or
to be less influenced by the choice of the technical parameters of the image acquisition

Typically:
coefficient α:
   Impact the reproducibility
   Optimize fracture prediction
coefficient β:
   Impact the contribution of the global mineralization of a (bone) tissue
coefficient γ:
   Influence the contribution of the overall geometric properties of a (bone) tissue
coefficient δ:
   Is linked to the signal/noise ratio of the image
coefficient ε:
   Impact the reproducibility
j) Finally at the end of the process, the bone texture score B is interpreted by comparison with age (FIG. 9), ethnicity, and gender matched controls.

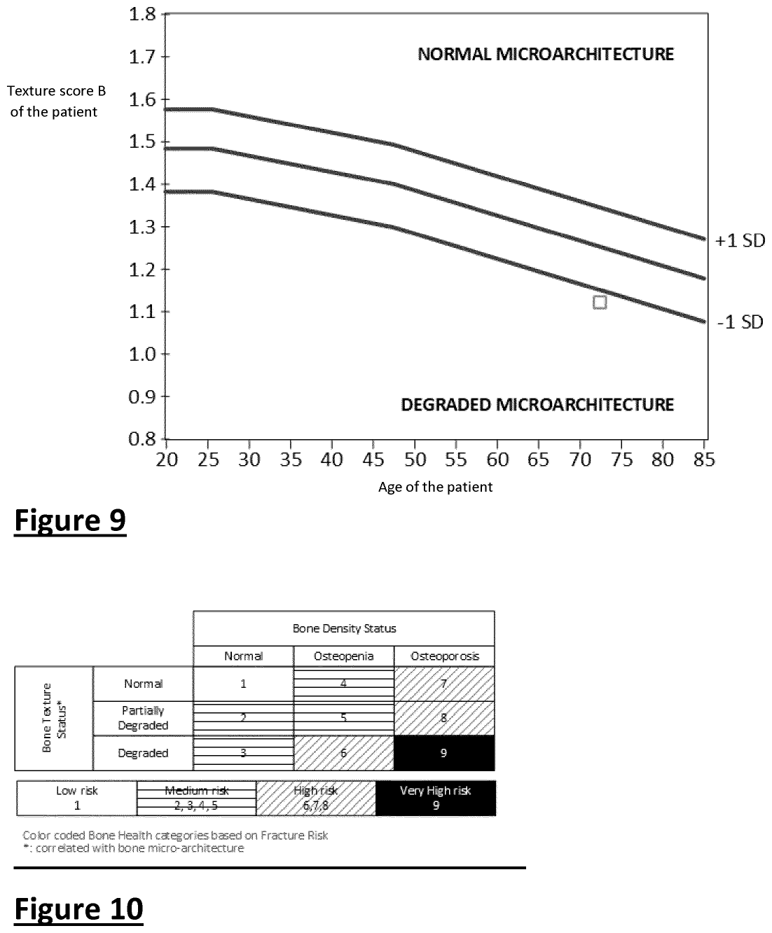

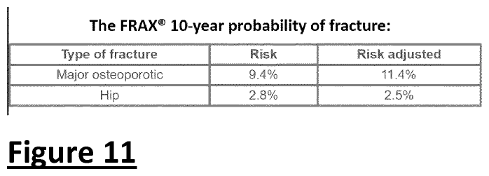

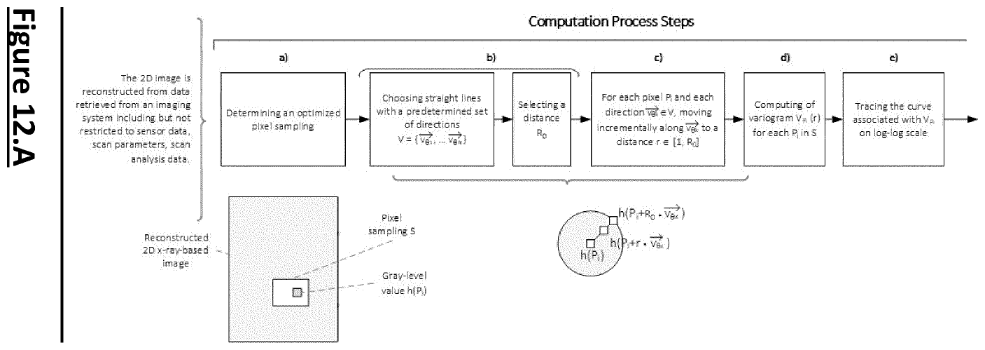

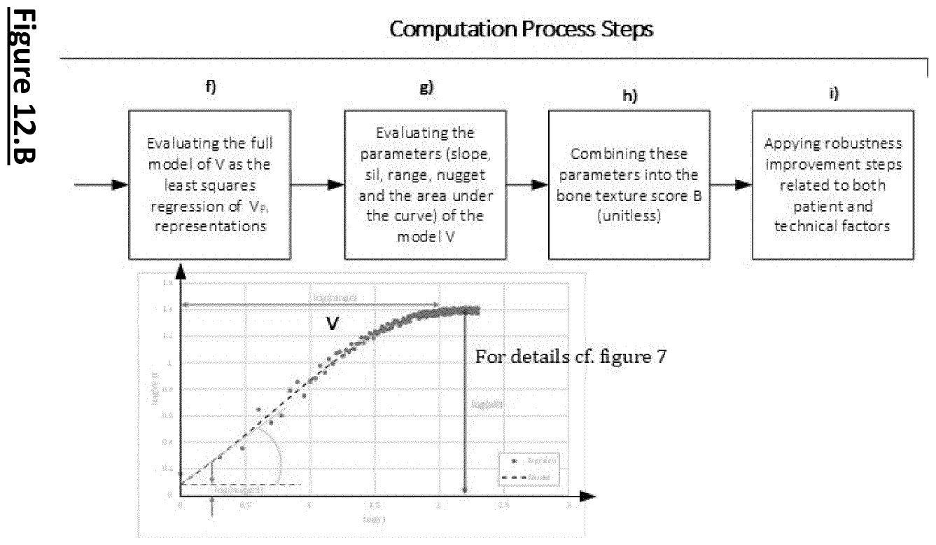

The patient's fragility fracture risk profile is also assessed as standalone or incorporated in probability model such as FRAX (FIG. 11) or combined with minimum BMD T-score (FIG. 10) of both spine and hip in a given clinical context or as a combination of BMD T-score and TBS categories to define the bone resilience index.

In the case of FIG. 9, to following parameters have been used or determined:
Bone image corresponds to FIG. 1,
Variogram corresponds to FIG. 7
Calculated a=0.73
Calculated b=1.35
Calculated c=2.0
Calculated d=0.08
Calculated e=1.30
B=αa+βb+γc+δd+εe=1.1 with
   α=1.6
   β=0.08
   γ=0.12
   δ=−0.97
   ε=−0.26
Considered technical or patient factors:
   Age=73 year old
   Weight=65 kg
   Size=1.66 m
   BMI=23.59
   Tissue Thickness=19 cm

α,β,γ,δ,ε are parameters used for giving respective weights between a, b, c, d, and/or e for calculating or determining the texture score B.

The embodiment of process according to the invention further comprises a step of displaying (on a screen):
determined sampling S on the image, and/or
chosen directions I on the image, and/or
computed variogram(s), and/or
evaluated parameter a, b, c, d and/or e, and/or
calculated texture score B, and/or
patient factors and/or technical factor(s) taken into account in the robustness improvement step.

Typically, at least one of the steps of the process according to the invention previously described, and more precisely each of the steps of the process according to the invention previously described is not performed in a purely abstract or purely intellectual manner but involves use of a technical means.

Typically, each of the steps a) to j) of the process according to the invention previously described is be implemented by technical means, preferably by at least one computer, one central processing or computing unit, one analogue electronic circuit (preferably dedicated), one digital electronic circuit (preferably dedicated) and/or one microprocessor (preferably dedicated) and/or by software means.

The following tables 1 and 2 are examples illustrating metric results of respectively univariate tests and logistic regression models which allow getting the best optimization of the distance Ro.

Univariate-Fracture group discrimination

TABLE 1
Univariate tests for the distance optimization

| | Texture score | Texture score | |
|---|---|---|---|
| | B Fractured group, | B Control group (Non | |
| | n = 559 n being the | fractured), n = 559 | |
| | number of patients in | (mean) n being the | P-value |
| Distance | this group | number of patients in | for |
| R₀ | (mean) | this group | comparison |
| 2 | 1.276 | 1.236 | 1.48e−3 |
| 4 | 1.272 | 1.236 | 5.91e−4 |
| 5 | 1.255 | 1.221 | 3.85e−4 |
| 6 | 1.164 | 1.131 | 2.24e−4 |
| 7 | 1.097 | 1.066 | 1.91e−4 |
| 8 | 1.032 | 1.002 | 1.79e−4 |

Technical factor optimization (Logistic model): example of compensation of low image resolution by high R₀

TABLE 2
Example of logistic model metrics tests
to obtain the best significancy

| | P-value significancy | |
|---|---|---|
| Distance | for B | |
| R₀ | significant if <0.05 | OR (Odds Ratio) |
| 2 | 0.6035 | 1.107 |
| 4 | 0.2595 | 1.241 |
| 5 | 0.1658 | 1.302 |
| 6 | 0.0965 | 1.372 |
| 7 | 0.0610 | 1.421 |
| 8 | 0.0463 | 1.465 |
| 9 | 0.0358 | 1.499 |
| 10 | 0.0307 | 1.519 |

Patient factors optimization: example of soft tissues:

logistic regression: INC FX^{˜}B (INC FX: Incident fracture)

TABLE 3
Summary of logistic regression results RAW versus adjusted

| | P-value significancy | |
|---|---|---|
| Variable | for B | OR (Odds Ratio) |
| B_raw | 0.1196 | 1.354 |
| B adjusted for | 0.0220 | 1.586 |
| soft tissues | | |

Table 3: On a given cohort, a logistic model allows the comparison between raw Texture Score B measurements versus patient factors corrected Texture Score B measurements.

In such model, we want to predict incident fracture i.e. INC_FX given such score measurements, raw versus adjusted.
Significance of the test statistic for patient factors corrected B (p-val=0.02) versus 0.12 for raw B
Odd ratio (per B increase) showing a better increase in the odds of incident fracture (from 35% to 58%)
Table 1: Calculus distance: On the same cohort:
   1) Case-control groups-fracture group versus control group:
      a. Showing increase of p-val for univariate analysis, case vs control Student tests, when distance increases from 2 to 10 px.
Table 2: Calculus distance: On the same cohort:
   1) Case-control groups #2-fracture incident group versus control group, logistic model, incident fracture ˜B (dist)
      a. Showing increase of p-val with increase of distance from 2 to 10
      b. Showing a better improvement in the odd per increase of B of the incident fracture with increment of distance from 2 to 10

The device according to the invention comprise technical means (in particular means arranged for and/or programmed to respectively calculate, determine, choose, compute, evaluate, combine, apply improvement step(s)) arranged and/or programmed to implement all the previously described steps (in particular the steps of respectively calculating, determining, choosing, computing, evaluating, combining, applying improvement step(s))

Typically, at least one of the means of the device according to the invention previously described, preferably each of the means of the device according to the invention (and in particular the means arranged for and/or programmed to calculate, determine, choose, compute, evaluate, combine, apply improvement step(s)), are technical means.

Typically, each of the means of the device according to the invention implementing the steps a) to j) previously described (and in particular the means arranged for and/or programmed to calculate, determine, choose, compute, evaluate, combine, apply improvement step(s)) comprise at least one computer, one central processing or computing unit, one analogue electronic circuit (preferably dedicated), one digital electronic circuit (preferably dedicated) and/or one microprocessor (preferably dedicated) and/or software means.

The device according to the invention comprises:
Means for implementing the step of acquiring the digitized image: these means for acquiring the digitized image typically comprise:
   conventional x-ray imaging system or
   digital x-ray imaging system, or
   Dual X-ray Absorptiometry (DXA) imaging system, or
   projected Computed Tomography (CT) imaging system, or
   Quantitative computed tomography (QCT) imaging system, or
   projected Quantitative computed tomography imaging system, or
   peripheral Quantitative computed tomography (pQCT) imaging system or
   High-Resolution peripheral Quantitative computed tomography (HR-pQCT) imaging system, and
means for implementing all the previously described steps a) to j): these means are typically grouped together in a single computer
a screen (arranged for displaying determined sampling S on the image, and/or chosen directions I on the image, and/or computed variogram(s), and/or evaluated parameter a, b, c, d and/or e, and/or calculated texture score B, and/or patient factors and/or technical factor(s) taken into account in the robustness improvement step).

Of course, the different characteristics, forms, variants and embodiments of the invention can be combined with each other in various combinations to the extent that they are not incompatible or mutually exclusive.

In the summary of the invention and detailed description of the figures and of realization modes of the invention the word “process” is used but it can be replaced in an equivalent way by the word “method”.

Of course, the invention is not limited to the examples which have just been described and numerous amendments can be made to these examples without exceeding the scope of the invention, as long as it stays in the scope of the claims.

1-36. (canceled)

37. A process for analyzing a texture of a human or animal tissue from a digitized image, obtained by X-Ray based imaging system, comprising a step of calculating, by technical means, at least one texture score B for the image by applying an experimental variogram to the tissue texture and applying, by the technical means, robustness improvement step that take into account, into the texture score B:
at least one patient factor related to the patient imaged on the image and/or
at least one technical factor related to the acquisition of the image.

38. The process according to claim 37, wherein the at least one patient factor comprises:
effect of patient morphology including at least one among:
effect of soft tissue, and/or tissue thickness, and/or its distribution and/or its composition in the patient, and/or indirect surrogates and/or
a weight and/or Body Mass Index (BMI) of the patient, and/or
a size of the patient, and/or
effect of at least one pathology or condition of the patient, and/or
effect of patient positioning during the acquisition of the image.

39. The process according to claim 37, wherein the at least one technical factor comprises:
potential defective detectors and sensors for acquiring the image, and/or effect of scan mode and settings for acquiring the image, and/or
technical characteristics of the imaging device which is used for acquiring the image effect of the variability in between imaging systems for acquiring the image, and/or the Signal-Noise-Ratio (SNR) of the image, and/or
the resolution of the image.

40. The process according to claim 37, wherein the tissue is a bone tissue, and the texture score B is a bone texture score B.

41. The process according to claim 40, wherein the digitized bi-dimensional image is chosen in a region having a trabecular structure.

42. The process according to claim 37, wherein the process comprises the following steps implemented by the technical means:
determining an optimized pixel sampling S of the image for which each pixel Pᵢ=(xᵢ,yᵢ)ϵS having its gray level value h(Pᵢ)
for at least one region of interest (ROI) of the pixel sampling S, choosing a predetermined set of directions I depending on the given region of interest (ROI),
for each pixel: computing at least one experimental variogram of the gray levels of the sampling S by moving from this pixel by at least one distance rϵ[1, R₀] along those directions I, an experimental variogram being computed for each predetermined direction or for all the predetermined directions at the same time,
for each experimental variogram of each pixel and/or for a global experimental variogram of the sampling S combining the experimental variograms for each pixel, evaluating at least one of the following parameters on a log-log scale:
the initial slope a,
the sill b, representing the asymptote value of the experimental variogram
the range c, representing the distance at which the experimental variogram curve transitions from a quasi-linear progression to an asymptotic behavior
the nugget d, representing the initial value of the experimental variogram, and
the area under the experimental variogram curve e,
combining:
the parameter(s) obtained for each pixel into a texture score B for each pixel, and/or
the parameter(s) obtained for sampling S into a texture score B for the sampling S, and/or
the parameter(s) obtained for each pixel into a texture score B for the sampling S
this step further comprising applying the robustness improvement step related to the at least one patient factor and/or the at least one technical factor into the texture score B:
by determining and/or correcting, as a function of patient and/or technical factor(s), and before or during the determination or calculation of score B, at least one parameter used for calculating or determining score B, preferably:
at least one parameter among R₀, a, b, c, d, and/or
at least one parameter (α,β,γ,δ,ε) used for giving respective weights between a, b, c, d, and/or e for calculating the texture score B
and/or
by correcting, as a function of patient and/or technical factor(s), score B.

43. The process according to claim 42, wherein the predetermined set of directions I depends on:
a skeletal site of a bone, the human or animal tissue being the bone, and/or
the region of interest (ROI), and/or
a resolution of the image, and/or
a signal/noise ratio of the image.

44. The process according to claim 42, wherein step of choosing predetermined set of directions I is done by determining:
a set of N directional vectors U={\vec{u_{θ}_{₁}}, . . . , {right arrow over (u_{θ}_{_{N}})}}, where θₖϵ[−π,π], ∀k∈1, . . . , N.

45. The process according to claim 44, wherein moving by a distance r is done by, for each pixel Pᵢ=(xᵢ,yᵢ)ϵS and each direction \vec{u_{θ}_{ₖ}}ϵU, moving along \vec{u_{θ}_{ₖ}} to a distance rϵ[1, R₀] in pixels, h(Pᵢ+r*\vec{u_{θ}_{ₖ}}) being the gray value of such pixel.

46. The process according to claim 44, wherein an experimental variogram is computed for all the predetermined directions at the same time, the step of computing the experimental variogram of the gray levels as a function of the distance r being done by averaging the squared differences of h over several pairs of pixels, each at distance r with the formula:
$${V}_{{P}_{i}} ( r ) = \frac{1}{N} * {\sum }_{k = 1}^{k = N} {[ h ( {P}_{i} ) - h ( {P}_{i} + {r}^{*} \vec{{u}_{{θ}_{k}}} ) ]}^{2} ,$$
V_{P}_{ᵢ}(r) being computed for every pixel PᵢϵS,

47. The process according to claim 42, wherein an experimental variogram is computed for each predetermined direction, h(O) being the gray level of an initial given pixel before moving, h(r) being the gray level of a given new pixel after moving by a distance r along one of the predetermined directions from the initial given pixel, the experimental variogram being computed with the formula: Vᵢ(r)=[h(r)−h(O)]² where iϵI.

48. The process according to claim 42, wherein each parameter a, b, c, d, and/or e is evaluated as a least squares regression model of the considered experimental variogram.

49. The process according to claim 42, wherein the parameters are combined into the texture score B using linear or nonlinear equations depending on a clinical context.

50. The process according to claim 42, wherein the process comprises for each experimental variogram of each pixel and/or for a global experimental variogram of the sampling S combining the experimental variograms for each pixel, evaluating at least one of the following parameters on a log-log scale:
the sill b, representing the asymptote value of the experimental variogram
the range c, representing the distance at which the experimental variogram curve transitions from a quasi-linear progression to an asymptotic behavior
the nugget d, representing the initial value of the experimental variogram, and
the area under the experimental variogram curve e.

51. The process according to claim 42, wherein the process comprises for each experimental variogram of each pixel and/or for a global experimental variogram of the sampling S combining the experimental variograms for each pixel, evaluating at least two of the following parameters on a log-log scale:
the initial slope a,
the sill b, representing the asymptote value of the experimental variogram
the range c, representing the distance at which the experimental variogram curve transitions from a quasi-linear progression to an asymptotic behavior
the nugget d, representing the initial value of the experimental variogram, and
the area under the experimental variogram curve e.

52. The process according to claim 42, wherein the process comprises for each experimental variogram of each pixel and/or for a global experimental variogram of the sampling S combining the experimental variograms for each pixel, evaluating the initial slope a and at least one of the following parameters on a log-log scale:
the sill b, representing the asymptote value of the experimental variogram
the range c, representing the distance at which the experimental variogram curve transitions from a quasi-linear progression to an asymptotic behavior
the nugget d, representing the initial value of the experimental variogram, and
the area under the curve e.

53. The process according to claim 37, wherein the texture score B is unitless.

54. A device for analyzing a texture of a human or animal tissue from a digitized image, obtained by X-Ray based imaging system, comprising means arranged to and/or programmed to calculate at least one texture score B for the image by applying an experimental experimental variogram to the tissue texture and means arranged to and/or programmed to apply robustness improvement step that take into account, into the texture score B:
at least one patient factor related to the patient imaged on the image and/or
at least one technical factor related to the acquisition of the image.

55. The device according to claim 54, wherein the at least one patient factor comprises:
effect of patient morphology including at least one among:
effect of soft tissue, and/or tissue thickness, and/or its distribution and/or its composition in the patient, and/or indirect surrogates and/or
a weight and/or Body Mass Index (BMI) of the patient, and/or
a size of the patient, and/or
effect of at least one pathology or condition of the patient, and/or
effect of patient positioning during the acquisition of the image.

56. The device according to claim 54, wherein the at least one technical factor comprises:
potential defective detectors and sensors for acquiring the image, and/or
effect of scan mode and settings for acquiring the image, and/or
technical characteristics of the imaging device which is used for acquiring the image
effect of the variability in between imaging systems for acquiring the image, and/or
the Signal-Noise-Ratio (SNR) of the image, and/or
the resolution of the image.

57. The device according to claim 54, wherein of the human tissue is a bone tissue, and the texture score B is a bone texture score B.

58. The device according to claim 57, wherein the digitized bi-dimensional image is an image imaging a trabecular structure.

59. The device according to claim 54, wherein the device comprises:
means arranged to and/or programmed to determine an optimized pixel sampling S of the image for which each pixel Pᵢ=(xᵢ,yᵢ)ϵS having its gray level value h(Pᵢ)
for at least one region of interest (ROI) of the pixel sampling S, means arranged to and/or programmed to choose a predetermined set of directions I depending on the given region of interest (ROI),
for each pixel: means arranged to and/or programmed to compute at least one experimental variogram of the gray levels of the sampling S by moving from this pixel by at least one distance rϵ[1, R₀] along those directions I, and arranged to and/or programmed to compute a experimental variogram for each predetermined direction or for all the predetermined directions at the same time,
for each experimental variogram of each pixel and/or for a global experimental variogram of the sampling S combining the experimental variograms for each pixel, means arranged to and/or programmed to evaluate at least one of the following parameters on a log-log scale:
the initial slope a,
the sill b, representing the asymptote value of the experimental variogram
the range c, representing the distance at which the experimental variogram curve transitions from a quasi-linear progression to an asymptotic behavior
the nugget d, representing the initial value of the experimental variogram, and
the area under the experimental variogram curve e,
means arranged to and/or programmed to combine:
the parameter(s) obtained for each pixel into a texture score B for each pixel, and/or
the parameter(s) obtained for sampling S into a texture score B for the sampling S, and/or
the parameter(s) obtained for each pixel into a texture score B for the sampling S
the device further comprising means arranged to and/or programmed to apply the robustness improvement step related to the at least one patient factor and/or the at least one technical factor into the texture score B:
by determining and/or correcting, as a function of patient and/or technical factor(s), and before or during the determination or calculation of score B, at least one parameter used for calculating or determining score B, preferably
at least one parameter among R₀, α, b, c, d, e, and/or
at least one parameter (α,β,γ,δ,ε) used for giving respective weights between a, b, c, d, and/or e for calculating the texture score B
and/or
by correcting, as a function of patient and/or technical factor(s), score B.

60. The device according to claim 59, wherein the predetermined set of directions I depends on:
a skeletal site of a bone, the human or animal tissue being the bone, and/or
the region of interest (ROI), and/or
a resolution of the image, and/or
a signal/noise ratio of the image.

61. The device according to claim 59, wherein the means arranged to and/or programmed to choose predetermined set of directions I are arranged to and/or programmed to choose predetermined set of directions I by determining:
a set of N directional vectors U={\vec{u_{θ}_{₁}}, . . . , {right arrow over (u_{θ}_{_{N}})}}, where θₖϵ[−π,π], ∀kϵ1, . . . , N.

62. The device according to claim 61, wherein the means arranged to and/or programmed to compute at least one experimental variogram are arranged to and/or programmed to compute at least one experimental variogram of the gray levels of the sampling S by moving by a distance r, for each pixel Pᵢ=(xᵢ,yᵢ)ϵS and each direction \vec{u_{θ}_{ₖ}}ϵU, along \vec{u_{θ}_{ₖ}} to a distance rϵ[1, R₀] in pixels, h(Pᵢ+r*\vec{u_{θ}_{ₖ}}) being the gray value of such pixel.

63. The device according to claim 61, wherein the means arranged to and/or programmed to compute at least one experimental variogram are arranged to and/or programmed to compute an experimental variogram for all the predetermined directions at the same time, the means arranged to and/or programmed to compute at least one experimental variogram being arranged to and/or programmed to compute the experimental variogram of the gray levels as a function of the distance r by averaging the squared differences of h over several pairs of pixels, each at distance r with the formula:
$${V}_{{P}_{i}} ( r ) = \frac{1}{N} * {\sum }_{k = 1}^{k = N} {[ h ( {P}_{i} ) - h ( {P}_{i} + {r}^{*} \vec{{u}_{{θ}_{k}}} ) ]}^{2} ,$$
V_{P}_{ᵢ}(r) being computed for every pixel PᵢϵS.

64. The device according to claim 59, wherein the means arranged to and/or programmed to compute at least one experimental variogram are arranged to and/or programmed to compute an experimental variogram for each predetermined direction, h(O) being the gray level of an initial given pixel before moving, h(r) being the gray level of a given new pixel after moving by a distance r along one of the predetermined directions from the initial given pixel, the means arranged to and/or programmed to compute at least one experimental variogram being arranged to and/or programmed to compute the experimental variogram with the formula: Vᵢ(r)=[h(r)−h(O)]² where iϵI.

65. The device according to claim 59, wherein the means arranged to and/or programmed to evaluate the parameters are arranged to and/or programmed to evaluate each parameter a, b, c, d, and/or e as a least squares regression model of the considered experimental variogram.

66. The device according to claim 59, wherein the means arranged to and/or programmed to combine the parameters are arranged to and/or programmed to combine the parameters into the texture score B using linear or nonlinear equations depending on a clinical context.

67. The device according to claim 59, wherein the means arranged to and/or programmed to evaluate the parameters are arranged and/or programmed, for each experimental variogram of each pixel and/or for a global experimental variogram of the sampling S combining the experimental variograms for each pixel, to evaluate at least one of the following parameters on a log-log scale:
the sill b, representing the asymptote value of the experimental variogram
the range c, representing the distance at which the experimental variogram curve transitions from a quasi-linear progression to an asymptotic behavior
the nugget d, representing the initial value of the experimental variogram, and
the area under the experimental variogram curve e.

68. The device according to claim 59, wherein the means arranged to and/or programmed to evaluate the parameters are arranged and/or programmed, for each experimental variogram of each pixel and/or for a global experimental variogram of the sampling S combining the experimental variograms for each pixel, to evaluate at least two of the following parameters on a log-log scale:
the initial slope a,
the sill b, representing the asymptote value of the experimental variogram
the range c, representing the distance at which the experimental variogram curve transitions from a quasi-linear progression to an asymptotic behavior
the nugget d, representing the initial value of the experimental variogram, and
the area under the experimental variogram curve e.

69. The device according to claim 59, wherein the means arranged to and/or programmed to evaluate the parameters are arranged and/or programmed, for each experimental variogram of each pixel and/or for a global experimental variogram of the sampling S combining the experimental variograms for each pixel, to evaluate the initial slope a and at least one of the following parameters on a log-log scale:
the sill b, representing the asymptote value of the experimental variogram
the range c, representing the distance at which the experimental variogram curve transitions from a quasi-linear progression to an asymptotic behavior
the nugget d, representing the initial value of the experimental variogram, and
the area under the experimental variogram curve e.

70. The device according to claim 54, wherein texture score B is unitless.

71. A computer program comprising instructions which, when executed in a computer, implement the steps of the process according to claim 37.

72. A computer-readable storage medium comprising instructions which, when executed by a computer, cause the computer to carry out the steps of the process according to claim 37.
